(19) United States
(12) Patent Application Publication
(10) Pub. No.: US 2016/0354101 A1
(43) Pub. Date: Dec. 8, 2016

(54) LAPAROSCOPIC MEDICAL DEVICE ASSEMBLY WITH DETACHABLE END EFFECTOR, AND METHOD OF USING SAME

(71) Applicant: Cook Medical Technologies LLC, Bloomington, IN (US)

(72) Inventor: Jeffry Scott Melsheimer, Springville, IN (US)

(21) Appl. No.: 15/058,600

(22) Filed: Mar. 2, 2016

Related U.S. Application Data
(60) Provisional application No. 62/169,636, filed on Jun. 2, 2015.

(51) Int. Cl. A61B 17/29; A61B 17/28

(52) U.S. Cl. CPC A61B 17/2909; A61B 17/282; A61B 2017/2808; A61B 2017/292; A61B 2017/2931

(57) ABSTRACT

A laparoscopic medical device assembly includes a laparoscopic tool with a handle actuator coupled to slide a deployment/capture device within, and relative to, a sheath. An end effector has a knob attached to a proximal end of an actuator rod that extends through a base, and includes a pair of arms pivotally attached to the base. The laparoscopic tool and the end effector are movable among a capture/unactuated configuration, a capture/actuated configuration and a release configuration. The end effector may be left clamped to tissue while the laparoscopic tool is removed allowing an alternative use of the trocar access.

### TECHNICAL FIELD

The present disclosure relates generally to medical devices utilized in laparoscopic procedures, and more particularly to an end effector that may be detached from, and later retrieved by, a laparoscopic tool.

### BACKGROUND

Often during laparoscopic surgery in the abdomen, there is need for multiple ports. For instance, a first port may be needed for visualization/insufflation/lighting, a second port may be utilized for the surgeons left hand tool, a third port for the surgeon's right hand tool, and possibly fourth and fifth ports for an assistant's left and right hand tools. All of these ports lead to many potential entry points for infection, many potential sources of pain, many potential scars, and can cause a potentially crowded work area.

The present disclosure is directed toward potentially relieving overcrowding and possibly enabling a greater number of tools relative to ports in a laparoscopic procedure.

### SUMMARY

In one aspect, a laparoscopic medical device assembly includes a laparoscopic tool with a handle actuator coupled to slide a deployment/capture device within, and relative to, a sheath. An end effector has a knob attached to a proximal end of an actuator rod extending through a base, and includes a pair of arms pivotally attached to the base and movable between a first shape and a second shape. The laparoscopic tool and the end effector are movable among a capture/unactuated configuration, a capture/actuated configuration and a release configuration. The pair of arms are in the second shape in the capture/unactuated configuration, and in the first shape in the capture/actuated configuration. In the release configuration, the end effector is out of contact with the laparoscopic tool.

In another aspect, an end effector for use with a laparoscopic tool as part of a laparoscopic medical device assembly includes a base that is too large to be received in a sheath of the laparoscopic tool. A first arm and a second arm are pivotally attached to the base and are movable between a first shape and a second shape. A biasing element is operably positioned to bias the first arm and the second arm toward the second shape. An actuator rod is slidably received through the base with a distal end positioned between the first arm and the second arm, and has a knob attached to a proximal end of the actuator rod. The knob is at a first distance from the base when the first arm and the second arm are in the second shape, and the knob is at a greater distance than the first distance from the base when the first arm and the second arm are in the first shape.

In still another aspect, a method includes moving the laparoscopic medical device assembly in a capture/unactuated configuration at a first location to a second location with the laparoscopic medical device assembly in a capture/actuated configuration. The laparoscopic medical device assembly is then changed from the capture/actuated configuration to a release configuration at the second location. The laparoscopic tool is moved out of contact with the end effector. The knob of the end effector is captured with the deployment/capture device at least in part by moving the laparoscopic tool back into contact with the end effector and changing from the release configuration to the capture/unactuated configuration. The laparoscopic medical device assembly is then changed from the capture/unactuated configuration to the capture actuated configuration, and the medical device assembly is moved to a third location.

### BRIEF DESCRIPTION OF THE DRAWINGS

FIG. 1 is a side view of a laparoscopic medical device assembly extending through a trocar while in a capture/unactuated configuration;

FIG. 2 is a side view of an end effector according to the present disclosure in a second shape;

FIG. 3 is a partially sectioned view of the end effector of FIG. 2 shown in its first shape;

FIG. 4 is a side view of the laparoscopic medical device assembly of the present disclosure in a release configuration;

FIG. 5 is a side view of the laparoscopic tool and the end effector separated from one another;

FIG. 6 is a side view of the laparoscopic medical device assembly in a capture/actuated configuration; and

FIG. 7 is a schematic view during a laparoscopic procedure in which two end effectors have been temporarily left in contact with different tissues within a patient.

### DETAILED DESCRIPTION

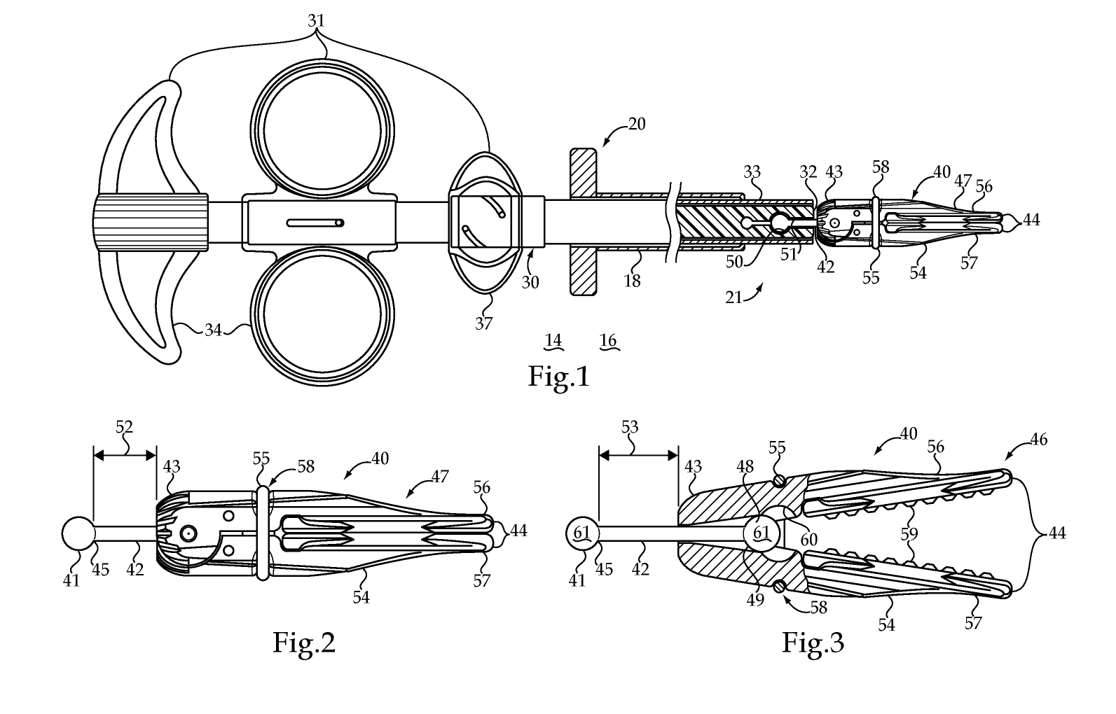

Referring to FIGS. 1-6, a laparoscopic medical device assembly 20 includes a laparoscopic tool 30 and a detachable end effector 40. Laparoscopic medical device assembly 20 may permit a physician to position one or more end effectors 40 in a patient and leave the end effector(s) 40 in place detached from a laparoscopic tool 30 while performing other aspects of a medical procedure. In other words, by withdrawing the laparoscopic tool 30 while leaving behind one or more end effectors 40, a trocar or other entry point can be utilized for another aspect of a procedure, or will allow for the use of fewer ports to perform a medical procedure. Thus, with the laparoscopic medical device assembly 20 of the present disclosure, an end effector 40 may retain its functionality, be introduced through a port and interact with a patient's tissue, such as clamp off a bleeding vessel. Then, the laparoscopic tool 30 can be detached from the end effector 40 allowing the end effector 40 to remain in place during a continuing portion of the procedure, then be reacquired and later withdrawn back out through the trocar when the medical procedure is finished.

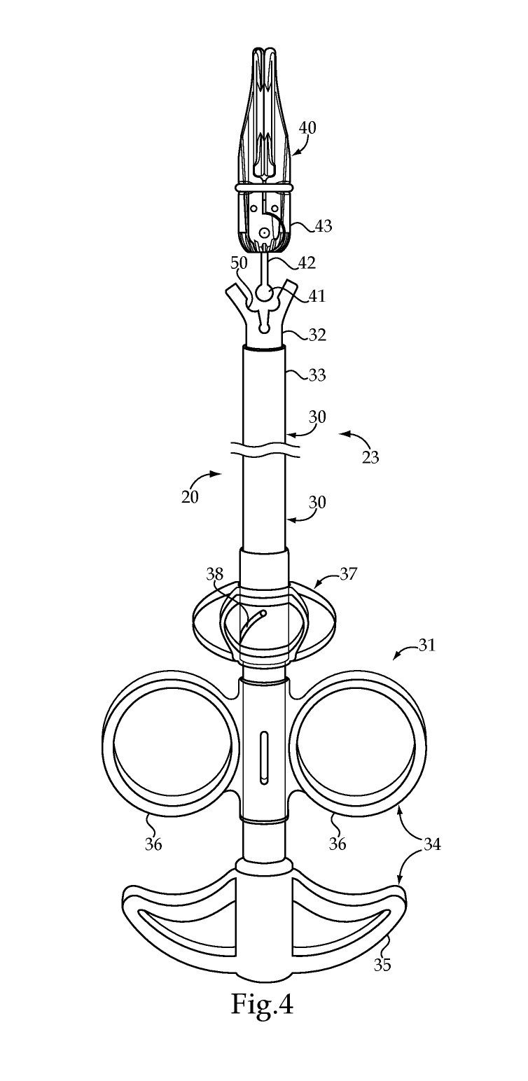

Laparoscopic tool 30 includes a handle actuator 31coupled to slide a deployment/capture device 32 within, and relative to, a sheath 33. The handle actuator 31 may include a shaft actuator 34 and a sheath positioner 37. The shaft actuator 34 may include a pair of finger rings 36 that are connected to move with the deployment/capture device 32, and a palm contact 35 attached to move with the outer sheath 33. Thus, movement of finger rings 36 toward palm contact 35 causes the deployment/capture device 32 to move proximally with respect to the distal end of the sheath 33. Finger rings 36 may be biased away from palm contact 35, such as by a spring positioned therebetween. Handle actuator 31 also includes a sheath positioner 37 that is operably coupled to sheath 33 via a pin and a spiral slot 38. Sheath positioner 37 may be rotated between a first position as shown in FIG. 4 in which the sheath 33 is retracted relative to finger rings 36, and a second position as shown in FIGS. 1, 5 and 6 in which sheath 33 is advanced distally with respect to finger rings 36. Those skilled in the art will appreciate that other hand manipulatable features known in the art could be substituted in place of the various features of handle actuator 31 without departing from the present disclosure. For instance, an alternative pistol grip structure with matching functions could be substituted in place for the in-line handle actuator 31 shown in the Figs. without departing from the present disclosure.

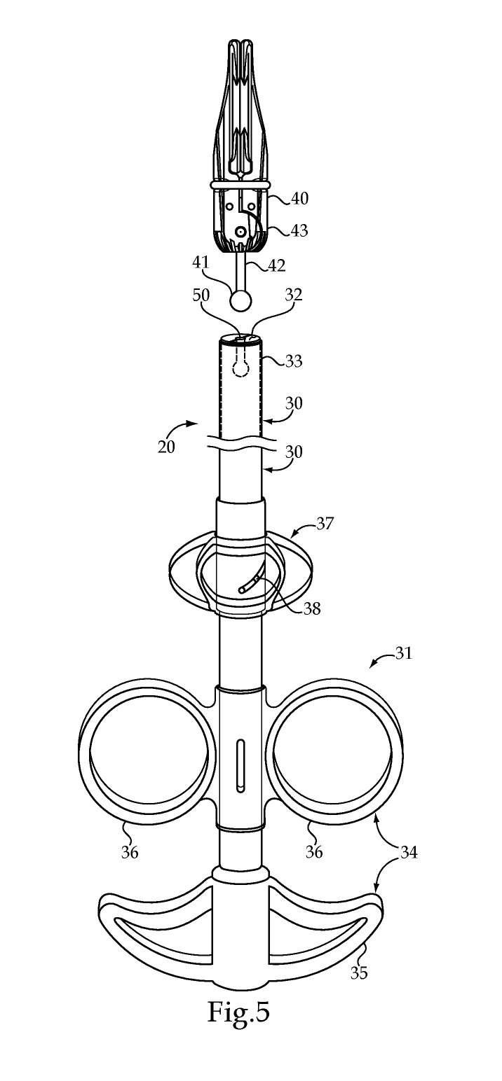

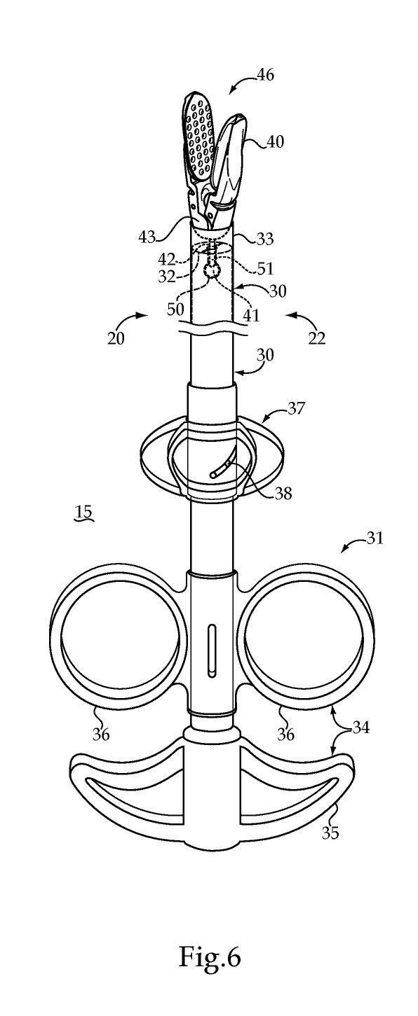

Like many laparoscopic tools, the sheath 33 may have a circular cross section and have a size and length appropriate for sliding through a trocar 18 so that end effector 40 can be appropriately positioned in the patient while handle actuator 31 is located outside of the patient in a conventional manner. The laparoscopic tool 30 and the end effector 40 are movable among a capture/unactuated configuration 21 as shown in FIG. 1, a capture/actuated configuration 22 shown in FIG. 6, and a release configuration 23 as shown in FIG. 4. FIG. 5 shows the laparoscopic tool 30 moving away from the end effector 40 after being released. Also as best shown in FIG. 4, the end effector 40 is out of contact with the laparoscopic tool 30 when in the release configuration 23. The knob 41 may be out of contact but still positioned between the open jaws of deployment/capture device 30 in the release configuration as shown in FIG. 4.

Referring now specifically to FIGS. 2 and 3, end effector 40 includes a knob 41 attached to a proximal end 45 of an actuator rod 42 that extends through, and is slidable with respect to, a base 43. End effector 40 also includes a pair of arms 44 pivotally attached to base 43 and movable between a first shape 46 and a second shape 47. In the illustrated embodiment, end effector 40 is illustrated as a grasper or a clamp 54. Pair of arms 44 are in the second shape 47 when the laparoscopic tool 30 and the end effector 40 are in the capture/unactuated configuration 21 as shown in FIG. 1. The pair of arms 44 are in the first shape 46 when the laparoscopic tool 30 and the end effector 40 are in the capture/actuated configuration as shown in FIG. 6. Assuming that the end effector 40 is not interacting with tissue or something else, the pair of arms 44 will be in the second shape 47 in the release configuration 23 as shown in FIG. 4.

The pair of arms 44 may be biased toward the second shape 47 using an appropriate biasing element 55 which takes the form of an elastic band 58 in the illustrated embodiment. Nevertheless, those skilled in the art will appreciate that other known structures could be utilized to bias pair of arms 44 between first shape 46 and the second shape 47 without departing from the present disclosure. For instance, if the end effector 40 took the form of a spreader, biasing element 55 could take an alternate form to bias the pair of arms 44 toward a spread configuration instead of as a clamp 54 as in the illustrated embodiment. Returning to the illustrated embodiment, actuator rod 42 may have a wedge 48 attached to a distal end 49 of the actuator rod 42, and the wedge 48 is operably positioned between the pair of arms 44. With this strategy, the laparoscopic tool 30 may tension actuator rod 42 to move knob 41 from a first distance 52 from base 43 to a second distance 53, which forces first arm 56 and second arm 57 of the pair of arms 44 to spread apart as shown in FIGS. 3 and 6. The second distance 53 is greater than the first distance 52. The tensioning of actuator rod 42 may be accomplished by forming base 43 of end effector 40 to be too large to be received in sheath 43 when deployment/capture device 32 is pulling knob 41 into laparoscopic tool 30 responsive to movement of shaft actuator 34.

The deployment/capture device 32 defines a receptacle 50 that is shaped to capture the knob 41 and an adjacent segment 51 of the actuator rod 42 in both the capture/unactuated configuration 21 shown in FIG. 1, and the capture/actuated configuration 22 shown in FIG. 6. The knob 41 may be considered to be trapped in the receptacle 50 in both the capture/unactuated configuration 21 and the actuated configuration 22. Although knob 41 is shown as having a spherical or ball shape, those skilled in the art will appreciate that a knob with numerous other shapes could work equally well. For instance, knobs 41 with an ovoid or other rounded atraumatic shape might be desirable. Other shapes could include spherical or ball shaped, egg shaped, pear shaped, teardrop shaped or even a door knob type shape and many other shapes without sharp edges could be utilized without departing from the intended scope of the present disclosure. In the illustrated embodiment, actuator rod 42 is symmetrical with identically shaped knobs 61 on opposite ends, which may allow for the actuator rod to be molded from a suitable plastic material integrally with the identically ball shaped knobs 61. Likewise, wedge 48 may take on a variety of shapes that interact appropriately with a wedge shaped cavity 60 in order to urge the pair of arms 44 apart when knob 41 is moved away from base 43 as shown in FIG. 3. In the illustrated embodiment actuator rod 42 is symmetrical with identical ball shaped knobs 61 on opposite ends, which allows end effector 40 to be manufactured and assembled without regard to which end of actuator rod 42 acts as a wedge 48 in which acts as a knob 41. Nevertheless, the wedge 48 and knob 41 could take on completely different shapes without departing from the present disclosure.

Although not necessary, the base 43 of end effector 40 may be out of contact with the sheath 33 of laparoscopic tool 30 in the capture/unactuated configuration 21 as shown in FIG. 1, but the base 43 may be in contact with the sheath 33 in the capture/actuated configuration as shown in FIG. 6. As stated earlier, the knob 41 is at a first distance 52 from the base 43 in both the capture/unactuated configuration 21 and the released configuration 23. On the otherhand, the knob 41 may be at a second distance 53, which is greater than the first distance 52, from the base 43 in the capture/actuated configuration 22. As stated earlier, end effector 40 is illustrated as a clamp 54 with the pair of arms 44 biased toward contact with each other by a biasing element 55 in the form of an elastic bend 58 that is in contact with each of the first arm 56 and the second arm 57. In other words, when the first arm 56 and the second arm 57 are configured as a clamp 54, opposing faces 59 may be in contact in the second shape 47 (FIG. 2), but be out of contact in the first shape 46, as best shown in FIG. 3. Elastic band 58 may be positioned in a groove defined by the respective arms 56 and 57 as best shown in FIGS. 2 and 3. Movement of wedge 48 toward base 43 against the action of biasing element 55 (elastic band 58) causes the laparoscopic medical device assembly 20 to responsively move from the capture/unactuated configuration 21 toward the capture/actuated configuration 22.

The deployment/capture device 32 may be made from a suitable semi-rigid material such as polyurethane that exhibits flexibility, some elasticity, and good tensile strength. It may be formed from a billet or rod of the material, which has been split into halves. One end of the two halves may be held together (as if still whole) while the receptacle 50 is formed in the free end. The receptacle 50 may be formed in the two halves of the opposite end and then the free or distal end may be curled open and heat set in a manner well known in the art. Once properly formed and heat set, the deployment/capture device 32 should be biased toward an open shape as shown in FIG. 4 when unconstrained by sheath 33. In other words, when deployment/capture device 32 is advanced beyond the distal end of sheath 33 by appropriately manipulating handle actuator 31, the deployment/capture device 32 will spread open in order to facilitate both capture and release of the knob 41 and actuator rod 42 of end effector 40. The actuator rod 42 and the associated knob 41 and wedge 48 may be molded or machined from a suitable polymer that is at least as strong as polyacetal, but maybe not any more lubricious that polyacetal. The actuator rod 42, if not molded integrally with knob 41 and maybe wedge 48, may be a flexible cable or strand as an alternative to the relatively rigid post of the illustrated embodiment. In any event, the actuator rod should be able to withstand a tensile load being imposed by the laparoscopic tool 30 when moving from the capture/unactuated configuration 21 to the capture/actuated configuration 22. Thus, in the context of the present disclosure, “actuator rod” should be interpreted to include flexible cables and the like.

It should be noted that when sheath positioner 37 is in the second position shown in FIG. 6, the laparoscopic medical device assembly 20 may only be capable of being moved between the capture/unactuated configuration 21 and the capture/actuated configuration 22. When the sheath positioner 37 is rotated to the first position shown in FIG. 4, the laparoscopic medical device assembly 20 may only be capable of moving between the capture/unactuated configuration 21 and the release configuration 23.

### INDUSTRIAL APPLICABILITY

The present disclosure finds generally applicability to medical procedures, and finds specific applicability for use in laparoscopic surgical procedures. Although the illustrated embodiment includes an end effector in the form of a clamp or a grasper, those skilled in the art will appreciate that other end effectors, such as a spreader would also fall within the intended scope of the present disclosure.

Although not necessary, the handle actuator 31 of the laparoscopic medical device assembly 20 is configured so that sheath positioner 37 is utilized to facilitate capture and release of knob 41 and hence end effector 40, whereas the shaft actuator 34 is utilized to actuate and de-actuate the end effector 40 in a manner consistent with the expected action of other laparoscopic medical devices. Thus, with the illustrated embodiment, one cannot go directly from the release configuration 23 of FIG. 4 to the capture/actuated configuration of FIG. 6 by manipulating only one of the shaft actuator 34 and the sheath positioner 37. In other words, in the illustrated embodiment, one must pass through the capture/unactuated configuration of FIG. 1 when moving between the release configuration 23 of FIG. 4 and the capture/actuated configuration 22 of FIG. 6, and vice versa.

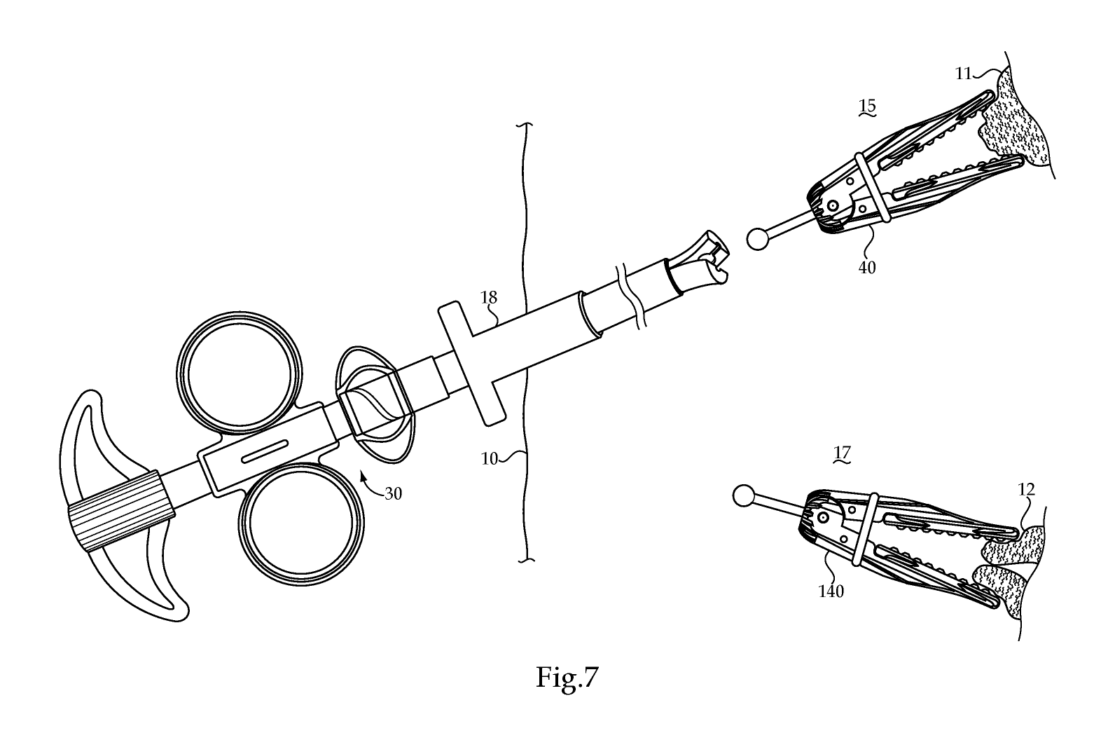

Referring now in addition to FIG. 7, a method according to the present disclosure may begin by moving the laparoscopic medical device assembly 20 in a capture/unactuated configuration 21 at a first location 14 (FIG. 1) to a second location 15 (FIGS. 6 and 7) with the laparoscopic medical device assembly in a capture/actuated position 22. For instance, the laparoscopic medical device assembly 20 shown in FIG. 1 may start outside of the patient's body 10, then be advanced through a trocar 18 into a patient's body 10 until arriving at a clamping location 15. During this portion of the procedure, the sheath positioner 37 may be “parked” at the second or advanced position so as to “lock” end effector 40 onto laparoscopic tool 30 so that it cannot become dislodged during introduction or withdrawal. In other words, the laparoscopic medical device assembly 20 may be incapable of achieving the release configuration 23 when the sheath positioner 37 is in the advanced or second position as shown in FIGS. 1, 5 and 6. As the laparoscopic medical device assembly 20 approaches location 15 where end effector 40 is to be deployed, shaft actuator 34 may be actuated by moving finger rings 36 toward palm contact 35, as shown in FIG. 6, so that base 43 contacts the distal end of sheath 33 while putting the actuator rod 42 in tension to spread arms 56 and 57 apart against the action of biasing element 55. When in this configuration the physician may position tissue 11 between arms 57 and 56. After this is done, the force on finger rings 36 and palm contact 35 can be relaxed to allow the internal bias to move them apart. This will also relieve tension on actuator rod 42 so that biasing element 55 can bias arms 56 and 57 toward each other to clamp the tissue 11 therebetween. Next, the sheath positioner 37 may be rotated from the second position to the first position to allow the deployment/capture device 32 to spread open to the release configuration 23. When this is done, the laparoscopic tool 30 can be moved out of contact with, and away from, end effector 40, which is left in place clamped to tissue 11 as shown in FIG. 7. After laparoscopic tool 30 and end effector 40 are separated, sheath positioner 37 may be rotated back to the second position to re-close deployment/actuator device 32 so that laparoscopic tool 30 can be more easily withdrawn through trocar 18. The laparoscopic tool 30 may then be completely withdrawn through trocar 18 so that the port may be used for another task during the remainder of the laparoscopic medical procedure, or the laparoscopic tool 30 may be loaded with a second end effector 140. For instance, a second end effector 140 may be delivered with laparoscopic tool 30 to contact tissue 12 at a fourth location 17. Later in the procedure, the laparoscopic tool 30 may be configured as shown in FIG. 4 and moved back toward contact to retrieve end effector 40. When properly positioned, the knob 41 can be captured with the deployment/capture device 32 by rotating the sheath positioner 37 from the first position (FIG. 4) to the second position (FIG. 1). In other words, capture is accomplished by changing the laparoscopic medical device assembly 20 from the release configuration 23 to the capture/unactuated configuration 21. Next, the laparoscopic medical device assembly 20 may be changed from the capture/unactuated configuration 21 to the capture/actuated configuration 22 in order to detach end effector 40 from tissue 11. This is accomplished by manipulated shaft actuator 35 to move finger rings 36 toward palm contact 35. The rejoined medical device assembly 20 may then be moved away from tissue 11. Next the force on shaft actuator is relaxed, the internal bias moves finger ringer 36 away from palm contact 35, the biasing element 58 moves end effector 40 to the second shape, and the complete assembly assumes the capture/unactuated configuration 21 as shown in FIG. 1. The laparoscopic medical device assembly 20 may then be moved to a third location 16, such as back out of trocar 18 outside of the persons body 10.

Thus, end effector 40 may be clamped to tissue 11, left in place during a procedure, and then later retrieved and removed from the patient leaving the port associated with trocar 18 available for other purposes when end effector 40 is left clamped to tissue 11. As discussed earlier, changing the laparoscopic medical device assembly 20 from the capture/unactuated configuration 21 to the capture/actuated configuration 22 includes pulling the knob 41 away from the base 43 of the end effector 40. As an example, end effector 40 may be utilized to clamp off a bleeding vessel in the abdomen 10 of the patient while other aspects of a laparoscopic surgical procedure are being performed through the port used to deliver the end effector 40, and maybe utilizing other ports in a manner known in the art.

The present description is for illustrative purposes only, and should not be construed to narrow the breadth of the present disclosure in any way. Thus, those skilled in the art will appreciate that various modification might be made to the presently disclosed embodiments without departing from the full and fair scope and spirit of the present disclosure. Other aspects, features and advantages will be apparent upon an examination of the attached drawings and appended claims.

what is claimed is:

1. A laparoscopic medical device assembly comprising:
a laparoscopic tool with a handle actuator coupled to slide a deployment/capture device within and relative to a sheath;
an end effector with a knob attached to a proximal end of an actuator rod extending through a base, and including a pair of arms pivotally attached to the base and being movable between a first shape and a second shape;
wherein the laparoscopic tool and the end effector are movable among a capture/unactuated configuration, a capture/actuated configuration and a release configuration; and
wherein the pair of arms are in the second shape in the capture/unactuated configuration, and in the first shape in the capture/actuated configuration, and the end effector is out of contact with the laparoscopic tool in the release configuration.

2. The laparoscopic medical device assembly of claim 1 wherein the pair of arms are biased toward the second shape; and
the laparoscopic tool tensions the actuator rod when moved from the capture/unactuated configuration to the capture/actuated configuration.

3. The laparoscopic medical device assembly of claim 2 wherein the actuator rod has a wedge attached to a distal end of the actuator rod, and the wedge is operably positioned between the pair of arms.

4. The laparoscopic medical device assembly of claim 1 wherein the deployment/capture device defines a receptacle shaped to capture the knob and an adjacent segment of the actuator rod in both the capture/unactuated configuration and the capture/actuated configuration; and
the knob is trapped in the receptacle in both the capture/unactuated configuration and the capture/actuated configuration.

5. The laparoscopic medical device assembly of claim 1 wherein the base of the end effector is too large to be received in the sheath;
the base is out of contact with the sheath in the capture/unactuated configuration, and in contact with the sheath in the capture/actuated configuration.

6. The laparoscopic medical device assembly of claim 1 wherein the knob is a first distance from the base in both the capture/unactuated configuration and the release configuration; and
the knob is a second distance, which is greater than the first distance, from the base in the capture/actuated configuration.

7. The laparoscopic medical device assembly of claim 1 wherein the end effector is a clamp with the pair of arms biased toward contact with each other by a biasing element;
the actuator rod has a wedge attached to a distal end, and the wedge is operably positioned between the pair of arms; and
the wedge is moved toward the base responsive to movement from the capture/unactuated configuration toward the capture/actuated configuration.

8. The laparoscopic medical device assembly of claim 7 wherein the deployment/capture device defines a receptacle shaped to capture the knob and an adjacent segment of the actuator rod in both the capture/unactuated configuration and the capture/actuated configuration, and the knob is trapped in the receptacle in both the capture/unactuated configuration and the capture/actuated configuration;
wherein the base of the end effector is too large to be received in the sheath, and the base is out of contact with the sheath in the capture/unactuated configuration, and in contact with the sheath in the capture/actuated configuration; and
wherein the knob is a first distance from the base in both the capture/unactuated configuration and the release configuration, and the knob is a second distance, which is greater than the first distance, from the base in the capture/actuated configuration.

9. An end effector for use with a laparoscopic tool as part of a laparoscopic medical device assembly, the end effector comprising:
a base that is too large to be received in a sheath of the laparoscopic tool;
a first arm and a second arm pivotally attached to the base and being movable between a first shape and a second shape;
a biasing element operably positioned to bias the first arm and the second arm toward the second shape;
a actuator rod slidably received through the base with a distal end positioned between the first arm and the second arm, and having a knob attached to a proximal end of the actuator rod;
the knob being a first distance from the base when the first arm and the second arm are in the second shape, and the knob being greater than the first distance from the base when the first arm and the second arm are in the first shape.

10. The end effector of claim 9 wherein the first arm and the second arm are configured as a clamp with opposing faces in contact in the second shape and out of contact in the first shape.

11. The end effector of claim 9 wherein the actuator rod is symmetrical with identically shaped knobs on opposite ends.

12. The end effector of claim 9 including a wedge attached to a distal end of the actuator rod and operably positioned between the first arm and the second arm.

13. The end effector of claim 9 wherein the biasing element includes an elastic band in contact with each of the first arm and the second arm.

14. The end effector of claim 9 wherein the actuator rod is symmetrical with identical ball shaped knobs on opposite ends;
the biasing element includes an elastic band in contact with each of the first arm and the second arm;
one of the ball shaped knobs is positioned in a wedge shaped cavity defined by the first arm and the send arm; and
the first arm and the second arm are configured as a clamp with opposing faces in contact in the second shape and out of contact in the first shape.

15. A method of using a laparoscopic medical device assembly that includes a laparoscopic tool with a handle actuator coupled to slide a deployment/capture device within and relative to a sheath; and an end effector with a knob attached to a proximal end of an actuator rod extending through a base, and including a pair of arms pivotally attached to the base and being movable between a first shape and a second shape, the method comprising the steps of:
moving the laparoscopic medical device assembly in a capture/unactuated configuration at a first location to a second location with the laparoscopic medical device assembly in a capture/actuated position;
changing the laparoscopic medical device assembly from the capture/actuated configuration to a release configuration at the second location;
moving the laparoscopic tool away from the end effector;
capturing the knob with the deployment/capture device at least in part by moving the laparoscopic tool back into contact with the end effector and changing from the release configuration to the capture/unactuated configuration; and
changing from the capture/unactuated configuration to the capture/actuated configuration, and then moving the laparoscopic medical device assembly to a third location.

16. The method of claim 15 wherein the step of moving the laparoscopic medical device assembly in a capture/unactuated configuration at a first location to a second location with the laparoscopic medical device assembly in a capture/actuated position includes:
moving the end effector through a trocar in the capture/unactuated configuration.

17. The method of claim 15 wherein the step of changing the laparoscopic medical device assembly from the capture/actuated configuration to a release configuration at the second location includes:
interacting with tissue with the end effector.

18. The method of claim 17 wherein the step of interacting with the tissue includes clamping the tissue with the end effector; and
the step of moving the laparoscopic tool away from the end effector includes leaving the end effector clamped to the tissue.

19. The method of claim 15 wherein the end effector is a first end effector; and
using the laparoscopic tool to deliver a second end effector to contact tissue at a fourth location.

20. The method of claim 15 wherein the changing the laparoscopic medical device assembly from the capture/unactuated configuration to the capture/actuated configuration includes pulling the knob away from the base.
